(19) United States
(12) Patent Application Publication
(10) Pub. No.: US 2006/0104997 A1
(43) Pub. Date: May 18, 2006

(54) Monoterpene compositions and uses thereof

(76) Inventors: Panayiotis P Constantinides, Gurnee, IL (US); Reena T Patil, Des Plaines, IL (US); Elijah M Bolotin, Buffalo Grove, IL (US); Likan Liang, Mundelein, IL (US)

Correspondence Address: KATTEN MUCHIN ROSENMAN LLP, 525 WEST MONROE STREET, CHICAGO, IL, 60661-3693, US

(21) Appl. No.: 10/498,566

(22) PCT Filed: Dec. 11, 2002
(86) PCT No.: PCT/US02/39545

§ 371 Date: Nov. 17, 2005

Related U.S. Application Data
(60) Provisional application No. 60/339,778, filed on Dec. 11, 2001.
(60) Provisional application No. 60/378,900, filed on May 7, 2002.

(51) Int. Cl. A61K 9/00; A61K 9/127; A61K 31/337; A61K 31/11; A61K 31/045

(52) U.S. Cl. 424/400; 424/450; 514/449; 514/729; 514/693

(57) ABSTRACT

The present invention relates to pharmaceutical compositions and methods for the mucosal and oral administration of monoterpenes and derivatives thereof. The compositions of this invention further comprise one or more surfactants and cosolvents and are in the form of self-emulsifying compositions. The compositions of the invention may further comprise water-insoluble therapeutic agents, vaccines and diagnostics. Such agents include but are not limited to taxanes, steroids, topoisomerase inhibitors such as etoposide and other water-insoluble or lipophilic drugs.

### BACKGROUND

1. Field of the Invention

The present invention relates to delivery systems for the mucosal and parenteral administration of biologically active molecules, including, but not limited to, therapeutic agents, vaccines, allergens, antigens and diagnostic agents. In particular, the present invention relates to self-emulsifying compositions which are preconcentrates of emulsions and microemulsions, comprising monoterpenes and derivatives thereof, surfactants, optional cosolvents, and one or more biologically active molecules, and methods of administering biologically active molecules to an animal utilizing said compositions. The compositions of the invention promote the absorption of biologically active molecules across epithelial barriers, preferably mucosal barriers. The compositions of the invention can be used therapeutically, diagnostically or cosmetically.

2. Background of the Invention

Lipid systems have been widely exploited for development of drug delivery vehicles and systems. Most of the lipid-based systems that have been developed for delivery of poorly water-soluble, or lipophilic, drugs do not provide a desired level of bioavailability. Biologically active agents, drugs, compounds and the like with little or no solubility in water are also referred to as lipophilic or hydrophobic and these terms are indistinguishable within the scope of the present invention. For many lipophilic drugs, there remains the need to find a carrier system that will enhance the bioavailability of such drugs in the GI tract. It has long been observed that the bioavailability of many lipophilic drugs can be improved if they are administered with food, emulsified in an oil or a mixture of oil and surfactants. Most lipid-based delivery systems for drugs with poor water solubility have been developed around the concept of delivery in a hydrophobic media.

Several lipophilic therapeutic compounds are insufficiently soluble in tri-glycerides and thus cannot be formulated solely in triglyceride oils. Organic solvents are sometimes useful to solubilize hydrophobic drugs, but are incompatible with other pharmaceutical excipients and oral administration devices. Therefore, other biocompatible hydrophobic solvents or cosolvents are needed to address lipophilic drugs with low solubility in triglycerides and surfactants.

Lipid-based delivery systems such as emulsion systems, microemulsions systems, and their self-emulsifying preconcentrates are based on the use of polar lipids and related amphiphilic surfactant molecules to control the interaction of hydrophobic molecules with water. In many cases, delivery systems for hydrophobic drugs have also required the inclusion of organic solvents that are water miscible in order to increase the molecular interactions between drugs and lipid or surfactant components.

Lipids and surfactants are differentiable from short and long chain hydrocarbons in that they are amphiphilic molecules, having both hydrophilic and hydrophobic moieties. Surfactants are conveniently classified on an empirical scale known as the hydrophile-lipophile balance (HLB) which runs from about 1 to about 45 and specifically from about 1 to about 20 for non-ionic surfactants. HLB values closer to 1 represent surfactants with more lipophilic character, while HLB values that are greater than about 10 represent more hydrophilic surfactants.

Lipid-based delivery systems may additionally incorporate absorption enhancers, such as the salicylates, bile salts and other surfactants, which increase the permeation of peptide, protein, and lipophilic molecules across epithelial barriers. A wide variety of amphiphilic molecules are known to behave as absorption enhancers. In addition, bile salts and salicylates, medium chain fatty acid salts and esters, and medium chain monoglycerides and di-glycerides are known to have mucosal absorption enhancing activity. Absorption enhancement with these molecules is attributed to the presence of medium chain C₆-C₁₂ fatty acyl chains (6-12 carbon atoms in length), particularly those esterified with C₈-C₁₀ fatty acids (8-10 carbon atoms in length). As suggested above, enhancing molecules may be involved in opening up channels or tight junctions between cells, allowing paracellular transport of co-administered molecules.

Oil-in-water (o/w) emulsions are also commonly formed from oil(s), surfactant(s), and an aqueous phase. Typically oils used that comprise drug delivery systems are made to solubilize lipophilic drugs to make them more effective and less toxic. Oils used in typical emulsions are any of a number of oils such as mineral, vegetable, animal, essential and synthetic oils, or mixtures thereof. In many cases oils rich in triglycerides, such as safflower oil, cottonseed oil, olive oil or soybean oil are used. In its simplest form, a triglyceride-containing formulation suitable for delivering hydrophobic therapeutic agents is an oil-in-water emulsion containing the therapeutic agent. Such emulsions contain the hydrophobic therapeutic agent solubilized in an oil phase that is dispersed in an aqueous environment with the aid of a surfactant or a combination of surfactants. Therefore, one approach to making suitable formulations of hydrophobic drugs is to solubilize a hydrophobic drug in an oil and to disperse this oil phase in an aqueous solution. Depending on whether an oil is a solid or liquid at the ambient temperature, the oil-in-water emulsion can be characterized as a solid lipid particulate. Surfactants are also required to form solid emulsions. And the same forces that operate in liquid oil phase also cause the precipitation of hydrophobic drugs at the interface of lipids with water upon short or long term storage and destabilize lipid particle suspension systems. The dispersion may be stabilized by emulsifying agents and provided in emulsion form. In a water milieu, drugs dissolved in the oil phase or the solid lipid core phase may be dispersed by mechanical force to create droplets or spheres suspended in the aqueous phase that are stable in storage as a pharmaceutical preparation.

The formation of a stable oil-in-water emulsion may be enhanced by the use of surfactants that form the interface between the strictly hydrophobic oil and water. Depending on the nature of the oil and one or more surfactants, either large droplets characteristic of oil-in-water emulsions or much smaller structures characteristic of microemulsions or micellar structures are formed. Further control over size of droplets or particles can be obtained by high pressure homogenization or similar shear forces. Lipid particles are typically formed at higher ambient temperatures to melt the hydrophobic components. These formulations may modulate the drug's pharmacokinetics/pharmacodynamics after intravascular or oral administration.

Lipophilic therapeutic agents, while poorly soluble in aqueous solution, may be sufficiently lipophilic such that therapeutically effective concentrations can be prepared in triglyceride-based solvents forming colloidal oil particles, with broad particle size distribution, ranging from several hundred nanometers to several microns in diameter. The tendency of triglyceride-based emulsions to agglomerate and phase separate presents problems of storage and handling, and increases the likelihood that pharmaceutical preparations of, triglyceride-based emulsions initially properly prepared will be in a less optimal, less effective, and poorly-characterized state upon ultimate administration to a patient.

Microemulsion systems are ternary or quaternary systems typically formed from an oil phase, a surfactant, and water. For example, U.S. Pat. No. 5,707,648 (S. H. Yiv) describes microemulsions that contain an oil phase, an aqueous phase, and a mixture of surfactants. The solubilization of one phase into another in a microemulsion system is affected by a balance of attractive and repulsive forces. Microemulsions are thermodynamically stable, such that the droplets will not coalesce and precipitate over time. The diameter of microemulsion droplets is in the range of 10 to 200 nanometers, while emulsion droplets are generally greater than a micron. The interface of microemulsion droplets can be considered as a monolayer of surfactant. A microemulsion can be characterized by the amount of the dispersed phase solubilized in the continuous phase. Microemulsions have traditionally been formed using, in addition to the components described above, a second surfactant, which are generally short chain alcohols, ethanol or butanol, glycols such as propylene glycol and polyethylene glycol, or medium chain alcohols, amines, or acids.

Additional strategies to enhance the bioavailability of hydrophobic drugs include methods to increase surface area of drug crystals and the co-inclusion of P-glycoprotein (PGP) inhibitors in formulations in an effort to increase absorption. Many drugs are substrates for the PGP, which acts as an efflux pump. As disclosed in U.S. Pat. No. 6,245,8,05 (Broder, S., K. L. Duchin, and S. Selim/Baker Norton Pharmaceuticals, Inc.), cyclosporin A may be used to enhance the bioavailability of hydrophobic drugs by inhibiting PGP. Additional compounds that are known inhibitors of PGP can also be used to enhance the bioavailability of lipophilic drugs. These include other PGP inhibitors such as cylosporin and cyclosporin analogues, surfactants such as poloxamers, polysorbates, ∝-tocopherol polyethylene glycol esters, as well as therapeutic agents known to affect the activity of PGP such as verapamil and ketoconazole.

Monoterpenes are naturally occurring compounds found in the essential oils of many plants including fruits, vegetables, and herbs. Some monoterpenes have been shown to have anti-neoplastic activity and are candidates for development as cancer chemotherapeutics. Dietary monoterpenes are able to prevent and to induce regression of various forms of cancer. For example, the addition of monoterpenes such as limonene and perillyl alcohol to experimental animal diets prevents mammary, liver, lung, and other cancers. In addition, dietary monoterpenes have been used to treat a variety of rodent cancers, including carcinogen-induced breast and pancreatic carcinomas. Furthermore, limonene and perillyl alcohol are effective therapeutic agents against advanced N-methyl-N-nitrosourea-induced rat mammary carcinoma, with perillyl alcohol having approximately 5-fold greater activity than limonene.

Based on screening against panels of cell lines in vitro, monoterpenes may be effective in treating or preventing neuroblastomas and leukemias. In mammalian cancer treatment models, perillyl alcohol has been shown to be effective in inducing tumor regression. U.S. Pat. No. 5,587,402 teaches methods for treating leukemia with perillyl alcohol by oral administration of perillyl alcohol. U.S. Pat. No. 5,414,019 teaches the use of perillyl alcohol to treat mammalian carcinomas also by oral administration. U.S. Pat. No. 5,470,877 describes the use of perillyl acid methyl ester to treat cancer by oral administration.

Monoterpenes affect a number of steps in oncogenesis, including both the initiation stage and the progression stages of cancer. Activities that have been attributed to monoterpenes include the induction of apoptosis, cell cycle arrest, the inhibition of post-translational modification of proteins that are involved in signal transduction, and differential gene regulation. One of the major effects in cell regulation by monoterpenes is the upregulation of TGF-β expression, leading to cell apoptosis through the inhibition of G₁ and activation of the oncogene product ras and other signals activated by TGF-β (Ariazi et al., 1999 Activation of the transforming growth factor-beta signaling pathway and induction of cytostasis and apoptosis in mammary carcinomas treated with the anticancer agent perillyl alcohol, Cancer Res. 59:1917-1928). Tumors undergoing monoterpene-induced regression also overexpress the mannose 6-phosphate/IGF II receptor family. In addition, limonene and perillyl alcohol have been shown to inhibit protein prenylation. Since farnesylation of ras protein is involved in oncogenic transformation, inhibition of protein prenylation may be one of the anti-tumor effects of limonene and perillyl alcohol in humans and in animals. Such activities may underlie the human anticancer activity of perillyl alcohol and limonene.

For conventional oral systems, the therapeutic agent is released into the GI tract within a short period of time, and plasma drug levels peak at a given time, usually within a few hours after dosing. A controlled release oral dosage form is designed to maintain drug levels at constant effective concentrations. Generally, controlled delivery of lipophilic drugs requires techniques different than those employed with hydrophilic drugs. Lipophilic drugs must be solubilized in order to be released in a controlled fashion.

Many systems for oral delivery of hydrophobic drugs are oil-based wherein the hydrophobic drug being is dissolved in an oil. However, the administration of a drug in oil alone is not advantageous because of the poor miscibility of the oil with the aqueous environment of the gastrointestinal tract.

The low solubility of many hydrophobic therapeutic agents in aqueous solution causes many formulations to fail to provide therapeutically effective doses where needed. For example paclitaxel is now indicated in treatment of ovarian, breast, non-small cell lung, and head and neck carcinomas. One of the difficulties in administering paclitaxel is that the drug is insoluble in water and must be injected intravenously as a 50:50 mixture of Cremophor-EL surfactant (polyoxyethylated castor oil, BASF Corporation). Unfortunately, this formulation leads to a relatively high incidence of major hypersensitivity reactions upon intravenous administration which has been attributed to the unusually high concentration of Cremophor-EL required to solubilize the paclitaxel.

In view of the limitations of the current therapeutic methods and formulations for the administration of hydrophobic therapeutic agents, such as many anticancer agents, in the treatment of diseases, including cancer, there remains a need for a more effective means to administer such non-water soluble therapeutic agents that increase absorption. In addition, in view of the limitation of the current therapeutic approaches for the administration of lipophilic drugs, including many anti-cancer agents, there remains a need for more effective means to administer non-water soluble therapeutic agents that do not require long administration periods.

### SUMMARY OF THE INVENTION

The present invention is directed to pharmaceutical compositions comprising a monoterprene or derivative thereof, one or more surfactants and optionally one or more cosolvents. The compositions may be in the form of an emulsion preconcentrate and may be self-emulsifying upon dilution in an aqueous solution or biological fluid. The composition may also be in the form of a micro-emulsion preconcentrate. In a preferred embodiment of the invention, the monoterprene or derivative thereof has anti-neoplastic activity and is selected from the group consisting of perrillyl alcohol, perillic acid, perillaldehyde and perillaldehyde methyl ester. The compositions of the invention may further comprise one or more therapeutic agents. Preferably, the therapeutic agents have aqueous solubility of less than 1 mg/ml and preferably less than 0.1 mg/ml.

Surfactants useful in the practice of the present invention may be emulsifying agents and may be selected from the group consisting of an alkyl glycerolphosphoryl choline, a polyoxyethylene polymer, a block copolymer of polyoxyethylene and polyoxyethylene and ethoxylated glycerol ester. In a preferred embodiment, perillyl alcohol is present at about 1% to about 50% of the total weight of the composition. In another preferred embodiment, perillyl alcohol is present at about 5% to about 40% total weight of the composition. While in another preferred embodiment, perillyl alcohol is present at about 5% to about 20% of the total weight of the composition. In still another preferred embodiment perillyl alcohol is present at about 5% to about 10% of the total weight of the composition. In another embodiment of the present invention, surfactant may be present at about 1% to about 75%, about 10% to about 60%, or about 20% to about 50% of the total weight of the composition. A preferred lipophilic therapeutic agent according to the present invention is an anti-cancer agent. Preferred anti-cancer agents include taxane or analogs of taxane, topoisomerase inhibitors, daunorubicin, doxorubicin, or derivatives thereof. A preferred taxane analog is paclitaxel. Preferably, paclitaxel comprises from about 1% to about 20% and more preferably from about 1% to about 10% of the total weight of the composition. Preferred topoisomerase inhibitors include etoposide, camptothecin, topotecan, or derivatives thereof. The compositions of the invention may further comprise inhibitor of P-glycoprotein. Preferred inhibitors of P-glycoprotein may be selected from the group consisting of cyclosporin A, ketoconazole, verapamilor, or derivatives thereof. Surfactants useful in the practice of the present invention may also be inhibitors of P-glycoprotein. Such surfactants include, but are not limited to, polyoxyethylene block copolymer, a polysorbate and α-tocopaherol-polyethylene glycol-succinate.

Co-solvents useful in the compositions of the present invention include but are not limited to polyhydric alcohols. Preferred polyhydric alcohols may be selected from the group consisting of glycerol, sorbitol, mannitol, ethylene glycol, propylene glycol, polyethylene glycol, and mixtures thereof. Preferred polyethylene glycols have an average molecular weight of between 100 and 10,000 daltons and preferably from about 100 to about 1,000 daltons. In another preferred embodiment, polyethylene glycol has an average molecular weight in the range of about 200 to about 600 daltons. The present invention also comprises methods for treating diseases, and preferably neoplastic diseases, using the compositions of the present invention.

The compositions of the present invention may be used to treat a patient by the oral administration of said composition in forms selected from the group consisting of a soft gelatin capsule, a hard gelatin capsule, an enteric coated capsule, with flavoring agents and taste masking agents, or a tablet.

### DETAILED DESCRIPTION OF THE INVENTION

The present invention provides improved compositions and methods for administering therapeutic agents with low solubility in aqueous solutions by using a self-microemulsifying formulation. The “self-microemulsifying formulation” of the present invention comprises a preconcentrate which emulsifies when mixed with an aqueous solvent and forms stable emulsions containing the therapeutic agent upon exposure to gastrointestinal fluids. The “preconcentrate” of the present invention solubilizes and stabilizes the therapeutic agent, such that the agent readily disperses into submicron emulsion droplets upon contact with gastrointestinal fluid and peristaltic agitation during intestinal transit.

Many systems for oral delivery of hydrophobic drugs are oil-based wherein a hydrophobic drug is dissolved in an oil. However, the administration of a drug in oil alone is not advantageous because of the poor miscibility of the oil within the aqueous environment of the gastrointestinal tract. The present invention provides a self-emulsifying system, or an emulsion preconcentrate system, for delivery of hydrophobic therapeutic agents to mammals. The emulsions and emulsion preconcentrates of this invention are also effective to treat or prevent disease states or for use in the diagnosis of disease. The emulsions and emulsion preconcentrates of this invention are effective in promoting the absorption of hydrophobic biologically active materials by mucosal tissues.

Also, in accordance with the present invention is a method of treating disease, such as cancer, using a drug delivery system for increasing the bioavailability of one or more hydrophobic drugs by emulsifying one or more hydrophobic agents with a self-microemulsifying preconcentrate comprising a monoterpene, and one or more surfactants.

The present invention provides a self-microemulsifying excipient formulation for increasing the bioavailability of poorly water-soluble drugs or pharmaceutical compositions is disclosed. The formulation generally includes a water immiscible monoterpene, a surfactant, and a hydrophilic co-surfactant. According to the present invention, a drug with poor solubility in water is dissolved in the self-microemulsifying excipient formulation. More than one drug or pharmaceutical ingredient and/or formulation at a time can be used according to the present invention to yield a desired pharmaceutical composition. Additionally poorly water-soluble drugs and/or pharmaceutical ingredients can be used in the practice of the present invention and can then be used in combination with other drugs and/or pharmaceutical ingredients which may or may not be poorly water-soluble.

One embodiment of the present invention comprises a preconcentrate formulation which comprises a water immiscible solvent, such as a monoterpene or monoterpene derivative, one or more surfactants, one or more hydrophobic therapeutic agents, and preferably a cosolvent miscible with said water immiscible solvent and water. A preferred monoterpene is perillyl alcohol. The formulation may further comprise pharmaceutically acceptable excipients. The bioavailability of non water-soluble drugs is enhanced by forming a preconcentrate that promotes self-emulsification when mixed with intestinal fluids.

In addition to the biological activities described above, monoterpenes have chemical characteristics suitable for the improved delivery of other lipophilic chemotherapeutics particularly in reference to antitumor activity. For example, perillyl alcohol is a liquid oil-like alcohol that is immiscible with water, but highly miscible with oils, lipids, and can solubilize non-water-soluble compounds. The present invention takes advantage of the property of monoterpenes to solubilize hydrophobic material by combining hydrophobic therapeutic agents in a single formulation with monoterpenes, such as perillyl alcohol. One embodiment of the present invention comprises formulations of emulsions which emulsions comprise solubilizing monoterpene agents, lipophilic substances, surfactants, and optionally cosolvents. The emulsions of the present invention may comprise a monoterpene, including but not limited to perillyl alcohol (1-hydroxymethyl-4-isopropenyl-1-cyclohexene), (R)-1-Methyl-4-(1-methylethenyl)cyclohexene (d-limonene), 1-methyl-4-hydroxypropyl-1-cyclohexene (α-terpineol), 1-chloro-1-cyclohexene, 3-chloro-1,4-dimethyl-1-cyclohexene, carveol, carvone, dihydrocarveol, dihydrocarvone, pulegone, isopulegol, menthol, menthone, terpinen-4-ol, sobrerol, limonene oxide, uroterpenol, perillaldehyde, dihydroperillic acid, dihydroperillic acid methyl ester, β-myrcene, perillic acid-8,9-OH, α-pinene, linalool, and perillic acid. A preferred monoterpene is perillyl alcohol.

In a preferred embodiment of the invention, perillyl alcohol is present in an amount of about 0.1% to about 30% by weight of the preconcentrate. In another preferred embodiment of the invention, perillyl alcohol is present in an amount of about 10% to about 50% by weight of the preconcentrate. In a more preferred embodiment of the invention, perillyl alcohol is present in an amount of about 10% to about 40% by weight of the preconcentrate. In an even more preferred embodiment of the invention, perillyl alcohol is present in an amount of about 10% to about 30% by weight of the preconcentrate. In yet a more preferred embodiment of the invention, perillyl alcohol is present in an amount of about 10% to about 20% by weight of the preconcentrate.

The preconcentrate according to the present invention also comprises a surfactant. Such surfactants include, but are not limited to egg yolk phospholipids, ethoxylated diacyl glycerol and dialkyl ether glycerol. Other surfactants useful in the practice of the present invention include alkylphosphoryl choline or alkylglycerophosphoryl choline and other lipid surfactants such as 1,2-dioctylglycero-3-phosphoryl choline, 1,2-ditetradecylglycero-3-phosphoryl choline, 1,2-dihexadecylglycero-3-phosphoryl choline, 1,2-dioctadecylglycero-3-phosphoryl choline, 1-hexadecyl-2tetradecylglycero-3-phosphoryl choline, 1-octadecyl-2-tetradecylglycero-3-phosphoryl choline, 1-tetradecyl-2-octadecylglycero-3-phosphoryl choline, 1-hexadecyl-2-octadecylglycero-3-phosphoryl choline, 1-2-dioctadecylglycero-3-phosphoryl choline, 1-octadecyl-2-hexadecylglycero-3-phosphoryl choline, 1-tetradecyl-2-hexadecylglycero-3-phosphoryl choline, 2,2-ditetradecyl-1-phosphoryl choline ethane, and 1-hexadecyltetradecylglycero-3-phosphoryl. Anionic surfactants, such as alkyl or aryl sulfates, sulfonates, carboxylates or phosphates, and cationic surfactants such as mono-, di-, tri- and tetraalkyl or aryl ammonium salts may also be used in the practice of the present invention. Non-ionic surfactants such as alkyl or aryl compounds, whose hydrophilic part consists of polyoxyethylene chains such as Pluronic F68 (MW=8,000), polysorbate 80 (Tween 80) sugar molecules, polyalcohol derivatives such as polyvinyl alcohol or other hydrophilic groups may also be used. Zwitterionic surfactants that have a combination of the anionic or cationic groups, and whose hydrophobic part consists of any other polymer, such as polyisobutylene or polypropylene oxides, may also be used. Mixtures of these surfactants may also be used as may other surfactants well known in the art. Anionic surfactants with HLB values preferably greater than about 10, such as alkyl or aryl sulfates, sulfonates, carboxylates or phosphates, and cationic surfactants with HLB values preferably greater than about 10, such as mono-, di-, tri- and tetraalkyl or aryl ammonium salts may also be used in the practice of the present invention.

In a preferred embodiment of this invention, the surfactant comprises nonionic surfactants with HLB values preferably less than about 20, such as alkyl or aryl compounds, whose hydrophilic part consists of polyoxyethylene chains, sugar molecules, polyalcohol derivatives or other hydrophilic groups may also be used. The surfactant of this invention has HLB values preferably from 1-45, more preferably from 3-30, and even more preferably from 3-20.

In a preferred embodiment of the invention, surfactant is present in an amount of about 20% to about 50% by weight of the preconcentrate. In a more preferred embodiment of the invention, surfactant is present in an amount of about 10% to about 40% by weight of the preconcentrate.

In addition to the above constituents, the preconcentrate of this invention may contain a second surfactant or “co-surfactant”. Such second surfactants include but are not limited to the surfactants described above, Labrasol (Gattefosse Corporation), which is comprised of a mixture of capric caprylic (C₈-C₁₀) mono- and di-glycerides triglycerides. Other preferred surfactants include mono-glycerides or hydrophilic derivatives thereof, di-glycerides or hydrophilic derivatives thereof or mixtures of mono-and di-glycerides and derivatives. Additional surfactants include but are not limited to long alkyl chain sulfonates/sulfates such as sodium dodecylbenzene sulfonate, sodium lauryl sulfate and dialkyl sodium sulfosuccinate, quaternary ammonium salts, fatty alcohols such as lauryl, cetyl, and steryl, glycerylesters, fatty acid esters, and polyoxyethylene derivatives thereof and polyoxyethylene or polyoxyethyle/polypropylene block co-polymers. Other useful emulsifying agents include Pluronic F68 (MW=8,000). The second surfactant of this invention has HLB values preferably from 5-20, more preferably from 5-15, and even more preferably from from 10-15.

In a preferred embodiment of the invention, a co-surfactant is present in an amount of about 1% to about 50% by weight of the preconcentrate. In a more preferred embodiment of the invention, a co-surfactant is present in an amount of about 10% to about 40% by weight of the preconcentrate.

Another embodiment of the present invention are preconcentrates comprising monoterpenes, preferably perillyl alcohol, a lipophilic therapeutic agent, and surfactant(s) and cosolvent. For example, perillyl alcohol and other monoterpene alcohols are able to effectively dissolve water insoluble compounds such as taxanes and taxane analogues, steroids, topoisomerase inhibitors such as etoposide and other water-insoluble or lipophilic drugs, and thus are useful in the preparation of the formulations of the present invention.

The preconcentrate according to the present invention also comprises a hydrophobic therapeutic agent. Such hydrophobic therapeutic agents include but are not limited to methotrexate, cis-platin and derivatives, vincristine, vinblastine, quinolone, ciprofloxacin, progesterone, daunorubicin, teniposide, estradiol, doxorubicin, epirubicin, and taxanes. Other hydrophobic therapeutic agents useful in the practice of the present invention include prostaglandins, amphotericin B, testosterone, beclomethasone and esters, vitamin E, cortisone, dexamethasone and esters, betamethasone valerete and other steroids, nifedipine, griseofulvin, cyclosporin, digoxin, itraconozole, carbamazepine, piroxicam, fluconazole, indomethacin, steroids, ibuprofen, diazepam, finasteride and diflunisal. Other therapeutic agents may also be used including antibiotics (antiviral, antibacterial, antihelminthic, antiplasmodial, or antimycotic), analgesics and local anesthetics, antidepressants, antipsychotics, sedatives, hypnotics, hormones, cytokines, vaccine adjuvants and antigens, immunosuppressive agents, vasodilators, antiarrhythmics, calcium antagonists, cardiac glycosides, oligonucleotides, oligopeptides, anti-emetics, and migraine therapeutics.

The preconcentrate according to the present invention may also comprise hydrophilic therapeutic molecules that can be derivatized with a hydrophobic compound. Upon derivatization with a hydrophobic compound, the hydrophilic molecule may be solubilized within the hydrophobic phase of the emulsion droplet. Methods of derivatization include but are not limited to conjugation of fatty acids through ester linkages to the amino terminal amino acid of a peptide or to epsilon amino groups of lysines resulting in esterification of acyl chains to proteins and peptides. Further, oligosaccharides and polysaccharides may be derivatized through available hydroxyl groups and both DNA and RNA may be selectively acylated using similar techniques. The derivatizing agent may include a number of hydrophobic acyl groups.

Hydrophilic therapeutic and bioactive molecules may also be physically associated with the surface of an emulsion droplet through ionic interactions. An emulsion droplet with a net positive surface charge may be made using amphipathic surfactants comprising positively charged fatty acid chains. Such positively charged emulsion droplets will readily adsorb nucleic acids and other negatively charged compounds to the surface of the droplet. Emulsion droplets with surface adsorbed DNA may be used for gene transfection vehicles in vitro and gene transfer agents for treating genetic diseases and for genetic vaccination.

In one embodiment of this invention, the emulsions may also comprise one or more solvents in addition to perillyl alcohol. Suitable solvents are glycerol, polythethylene glycols, propylene glycol, sorbitol, mannitol, ethylene glycol or mixtures thereof.

In addition to the above constituents, the preconcentrate of this invention may contain a cosolvent, which is miscible in the hydrophobic phase in order to solubilize the hydrophobic therapeutic agent. Such cosolvents include but are not limited to polyethylene glycol and related esters of fatty acids, polymerizable fatty acids, or polymerizable lipids, and monoterpene alcohols such as perillyl alcohol or limonene.

In addition to the above constituents, the preconcentrate of this invention may contain other pharmaceutically acceptable compounds or excipients to increase the stability of the emulsion. Such pharmaceutically acceptable compounds or excipients include but are not limited to tragacanth, cetyl alcohol, stearic acid, and/or beeswax (Remington's Pharmaceutical Sciences, 1975).

A more preferred embodiment of the present invention includes a preconcentrate formulation which comprises a water immiscible solvent, preferably a monoterpene alcohol with anti-cancer activity such as perillyl alcohol, one or more surfactants,.one or more hydrophobic anti-cancer agents, and preferably a cosolvent miscible with said water immiscible solvent and water.

Preconcentrates of this more preferred embodiment are useful for the co-administration of lipophilic anti-tumor agents. Simultaneous delivery of anti-tumor agents increases the benefit over monotherapies, and thus more effectively treats tumors. Furthermore, the combination of hydrophobic anti-tumor agents and monoterpenes with anti-cancer activity, in particular perillyl alcohol, may lead to synergistic anti-cancer activities.

One of the preferred anti-cancer agents useful in the emulsions of the present invention is etoposide. Another of the preferred anti-cancer agents useful in the emulsions of the present invention is doxirubicin. Other preferred anti-cancer agents include daunorubicin, irenotecan, mitomycin, bleomycin, procarbazine, altretamine, and lipophilic pro-drug derivatives of methotrexate, hydrophobic cis-platin derivatives such as 2-hydrazino-4,5-dihydro-1H-imidazole with platinum chloride or 5-hydrazino-3,4-dihydro-2H-pyrrole with platinum chloride, vincristine, vinblastine, teniposide, epirubicin, camptothecin, teniposide, topotecan, etoposide, teniposide, monophosphoryl Lipid A, and muramyl dipeptide derivatives. Still other preferred anti-cancer agents useful in the practice of the present invention are taxanes, including but not limited to lipid-soluble taxane and taxane derivatives including paclitaxel (Taxol); docetaxel (Taxotere); spicatin; taxane-2, 13-dione, 5β-, 9β-, 10β-trihydroxy-, cyclic 9, 10-acetal; taxane-2, 13-dione, 5β, 9β, 10β-trihydroxy-, cyclic 9, 10-acetal; taxane-2β-, 5β-, 9β-, 10β-tetrol, cyclic 9, 10-acetal; cephalomannine-7-xyloside; 7-epi-10-deacetylcephalomannine; 10-deacetylcephalomannine; cephalomannine; taxol B; 13-(2′,3′-dihydroxy-3′-phenylpropionyl) baccatin III; yunnanxol; 7-(4-Azidobenzoyl)baccatin III; N-debenzoyltaxol A; O-acetylbaccatin IV; 7-(triethylsilyl)baccatin III; 7, 10-Di-O-[(2,2,2-trichloroethoxy)carbonyl]baccatin III; baccatin III 13-O-acetate; baccatin diacetate; baccatin; baccatin VII; baccatin VI; baccatin IV; 7-epi-baccatin III; baccatin V; baccatin I; baccatin III; baccatin A; 10-deacetyl-7-epitaxol; epitaxol; 10-deacetyltaxol C; 10-deacetyl-7-xylotaxol; 7-epi-10-deacetyltaxol; 10-deacetyltaxol; taxagafine and 10-deacetyltaxol B.

In a preferred embodiment of this invention, paclitaxel is present in an amount of about 0.1% to about 20% by weight of the preconcentrate, while perillyl alcohol is present in an amount of from about 1% to about 30% of the weight of the preconcentrate and the surfactant is present in an amount of about 0.5% to about 5% by weight of the preconcentrate.

A more preferred embodiment of the present invention includes a preconcentrate formulation which comprises perillyl alcohol, one or more surfactants, and preferably a cosolvent miscible with perillyl alcohol and water.

Other therapeutic uses of perillyl alcohol could be realized by development of effective self-microemulsifying drug delivery systems with therapeutic levels of perillyl alcohol or its derivatives. For example, as described in U.S. Pat. No. 6,133,324, perillyl alcohol and it derivative by themselves or in combination with immunosuppressive agents may be used to treat organ transplant patients to reduce the possibility of allograft rejection of the transplanted organ. Other uses include treatment of bacterial and fungal infections.

Another embodiment of the present inventions are effective self-microemulsifying drug delivery systems comprising monoterpenes, preferably perillyl alcohol, a lipophilic anti-cancer agent, surfactants and an aqueous phase for use in methods for treating cancer.

Yet another embodiment of the present invention are effective self-microemulsifying drug delivery systems comprising monoterpenes, preferably perillyl alcohol, surfactants and an aqueous phase for the treatment of bacterial infections. Yet another embodiment of the present invention are effective self-microemulsifying drug delivery systems comprising monoterpenes, preferably perillyl alcohol, surfactants and an aqueous phase for the treatment of fungal infections. Still another embodiment of the present invention are effective self-microemulsifying drug delivery systems comprising monoterpenes, preferably perillyl alcohol, surfactants and an aqueous phase for the treatment of arterial plaques.

Another embodiment of the present invention comprises a vaccine preconcentrate formulation which comprises a water immiscible solvent, such as a monoterpene or monoterpene derivative, one or more surfactants, one or more hydrophobic antigens, preferably a peptide, polypeptide, or protein, and preferably a cosolvent miscible with said water immiscible solvent and water, and preferably a liquid carrier or adjuvant. Suitable adjuvants include but are not limited to mineral gels, such as aluminum hydroxide, surface active substances such as lysolecithin or pluronic polyols, polyanions, peptides, oil emulsions, alum, Lipid A and derivatives of Lipid A such as monophosphoryl Lipid A (MPLA), cytokines, and lipophilic derivatives of muramyl dipetide (MDP). The preferred monoterpene is perillyl alcohol. The vaccine preconcentrate of the present invention comprise an effective immunizing amount of one or more antigens and a pharmaceutically acceptable carrier or excipient. Pharmaceutically acceptable carriers are well known in the art and include but are not limited to saline, buffered saline, dextrose, water, glycerol, sterile isotonic aqueous buffer, and combinations thereof. A further example of physiologically acceptable carrier is a physiologically balanced salt solution containing one or more stabilizing agents including but not limited to stabilized, hydrolyzed proteins and lactose. The pharmaceutically acceptable carrier is preferably sterile.

The preconcentrates of the present invention may be in the form of a liquid solution, suspension, emulsion, sustained release formulation, powder, and preferably solid forms such as capsules, tablets or pills. Preconcentrates for oral administration preferably include standard carriers including but not limited to pharmaceutical grades of mannitol, lactose, starch, magnesium stearate, sodium saccharine, cellulose, or magnesium carbonate. Additionally, the preconcentrates can contain tastemasking agents or can be administered with carriers containing tastemasking agents.

Preconcentrates in liquid form may be provided in a hermetically sealed container such as an ampoule or a sachet. The preconcentrate formulations are generally stored at 4° C. prior to use. The precise dose of therapeutically effective agent(s) in the preconcentrate will depend on the route of administration, and the nature of the patient, and should be decided according to the judgment of the practitioner and each patient's circumstances according to standard clinical techniques.

Another embodiment of the present invention are methods for treating a disease, such as cancer, using a self-microemulsifying drug delivery system comprising administering to a mammal a preconcentrate in a suitable oral dosage form, such as a soft or hard-filled gelatin capsule, wherein said preconcentrate comprises a water immiscible solvent, such as a monoterpene, one or more surfactants, one or more hydrophobic therapeutic agents, and preferably a cosolvent miscible with said water immiscible solvent and water, whereby said preconcentrate mixes with an aqueous solvent, preferably gastrointestinal fluids, and which forms stable dispersions containing the hydrophobic therapeutic agent. The present invention improves the bioavailability of a non water-soluble drugs by forming a preconcentrate that promotes self-emulsification when mixed with an aqueous solvent. The stable dispersions of the current invention are preferably from about 50 nm to about 1 μm.

In a preferred embodiment of the invention, the monoterpene is present in an amount of about 10% to 50% by weight of the preconcentrate. More preferably, the monoterpene is present in an amount of about 10%-50% by weight of the preconcentrate.

In another preferred embodiment of the invention, the surfactant, as described above, is present in an amount of about 5% to 90% by weight of the preconcentrate. More preferably, the surfactant is present in an amount of about 10% to 75% by weight of the preconcentrate and most preferably from about 10% to 50% by weight of the preconcentrate.

In another preferred embodiment of the invention, a second surfactant, as described above, is present in an amount of about 5% to 90% by weight of the preconcentrate. More preferably, a second surfactant is present in an amount of about 5% to 50% by weight of the preconcentrate.

It is another objective of this invention to provide combination therapy for cancer by incorporating lipid insoluble anti-cancer drugs directly in the perillyl alcohol. Among these are lipophilic therapeutic agents which are soluble in perillyl alcohol. Representative lipophilic drugs include but are not limited to methotrexate, cis-platin and derivatives, vincristine, vinblastine, quinolone, ciprofloxacin, progesterone, teniposide, estradiol, doxorubicin, epirubicin, and taxanes.

Other lipophilic drugs that can be used include prostaglandins, amphotericin B, testosterone, beclomethasone and esters, vitamin E, cortisone, dexamethasone and esters, betamethasone valerate and other steroids. The lipophilic, poorly water-soluble active drug or pharmaceutical ingredient utilized in accordance with the present invention include but are not limited to nifedipine, griseofulvin, cyclosporin, digoxin, itraconozole, carbamazepine, piroxicam, fluconazole, indomethacin, steroids, ibuprofen, diazepam, finasteride, and diflunisal, for example. Other pharmaceutical ingredients or other drugs which are lipophilic or poorly water-soluble can also be used in accordance with the present invention.

A more preferred embodiment of the present invention includes methods for treating cancer, using a self-microemulsifying drug delivery system comprising administering to a mammal with cancer a preconcentrate in a suitable oral dosage form, such as a soft or hard-filled gelatin capsule, wherein said preconcentrate comprises a water immiscible solvent, preferably a monoterpene alcohol with anti-cancer activity such as perillyl alcohol, one or more surfactants, one or more hydrophobic anti-cancer agents, and preferably a cosolvent miscible with said water immiscible solvent and water, whereby said preconcentrate mixes with an aqueous solvent, preferably gastrointestinal fluids, and which forms stable dispersions containing said hydrophobic anti-cancer agent. The preferred hydrophobic anti-cancer agents are described above.

The present invention improves the bioavailability of lipophilic anti-cancer drugs by forming a preconcentrate that promotes self-emulsification when mixed with an aqueous solvent or intestinal fluids. The emulsion preconcentrates of the current invention form emulsion droplets that are preferably from about 1 nm to about 5 μm and more preferably from about 10 nm to 1 μm when dispersed in water or in contact with gastrointestinal fluid.

In a preferred embodiment of this invention, the composition includes perillyl alcohol, water and a mixture of one or more hydrophilic or lipophilic surfactants.

In a preferred embodiment of the invention, perillyl alcohol is present in an amount of about 1% to about 40% by weight of the preconcentrate. In another preferred embodiment of the invention, perillyl alcohol is present in an amount of about 5% to about 40% by weight of the preconcentrate. In a more preferred embodiment of the invention, perillyl alcohol is present in an amount of about 5% to about 20% by weight of the preconcentrate. In an even more preferred embodiment of the invention, perillyl alcohol is present in an amount of about 10% to about 30% by weight of the preconcentrate. In yet a more preferred embodiment of the invention, perillyl alcohol is present in an amount of about 10% to about 20% by weight of the preconcentrate.

In a preferred embodiment of this invention, paclitaxel is present in an amount of about 0.1% to about 20% by weight of the preconcentrate, while perillyl alcohol is present in an amount of from about 1% to about 50% of the weight of the preconcentrate and the surfactant is present in an amount of about 0.5% to about 50% by weight of the preconcentrate.

In a preferred embodiment of this invention, the preconcentrate is prepared by dissolving paclitaxel or other taxanes in perillyl alcohol, then adding emulsifiers described above, and preferably surfactants, solvents, additives, and preservatives, followed by sterile filtration through polycarbonate filters and dispensing into a suitable dosage form, preferably vials or gelatin capsules.

A more preferred embodiment of the present invention includes methods for treating disease, using a self-microemulsifying drug delivery system comprising administering to a mammal a preconcentrate in a suitable oral dosage form, such as a soft or hard-filled gelatin capsule, wherein said preconcentrate comprises perillyl alcohol, one or more surfactants, and preferably a cosolvent miscible with said water immiscible solvent and water, whereby said preconcentrate mixes with an aqueous solvent, preferably gastrointestinal fluids, and which forms stable dispersions containing perillyl alcohol.

The preconcentrates of the present invention are preferably administered through mucosal tissue or epithelia. The preconcentrate compositions of the invention can be delivered orally in the form of tablets, capsules, cachets, gelcaps, solutions, suspensions, or topically in the form of creams, ointments, suppositories and the like.

The preconcentrates of the present invention are therefore administered by those routes which optimize uptake by mucosa, such as sublingual, buccal, rectal and intranasal, and preferably oral. The preconcentrates of the present invention may also be administered by such routes as topical, transdermal and parenteral.

If administered topically the preconcentrate is preferably administered in the form of an ointment or transdermal patch. If administered intranasally the preconcentrate is preferably administered in an aerosol form, spray, mist or in the form of drops. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

The preconcentrates and emulsion compositions of the present invention are suitable for administration to animals, preferably mammals and birds, and more preferably humans. For example, domestic animals such as dogs and cats, as well as domesticated herds, cattle, sheep, pigs and other domesticated mammals may be treated or vaccinated with the preconcentrates of the present invention. In a preferred embodiment, the preconcentrates of the present invention are administered to humans. Preconcentrates and emulsion compositions are preferably provided in a hermetically sealed container such as an ampoule or sachet, and stored at 4° C. Dosages of the preconcentrates and emulsions compositions will vary depending on the individual patient and the mode of administration. Such dosages can be determined by a skilled physician using standard techniques.

The development of an oral formulation for an insoluble or poorly soluble drug often involves the designing of a system that will affect the pH of the micro-environment surrounding the drug form in the GI tract after ingestion. In particular, the formulation may contain disintegrants and/or other agents that work to increase or decrease the pH of the micro-environment, and thus enhance drug dissolution. In addition, the drug may also be granulated to reduce its particle size and/or increase the surface area that is exposed to the gastric fluid. The amount of exposed surface area will affect the rate of drug dissolution and thus the amount of active drug that will be absorbed by the patient. With respect to drug compounds of very poor or limited solubility, those skilled in the art have used cosolvents, surfactants or wetting agents to reduce the surface tension of the liquid environment of the gastric fluid in which the active drug is to be dissolved. These agents wet the active drug more quickly so that more of the drug is exposed to the gastric fluid in a shorter time, and may enhance its dissolution. Common types of surfactants and cosolvents that can be used include the cationic, anionic (e.g., sodium lauryl sulfate and gelatin), and nonionic (e.g., Myrj) types, as well as such cosolvents as the polyethylene glycols (PEGs). The role of the binder in the tablet drug form is to provide a tablet with sufficient hardness and integrity, but also must allow for sufficient disintegration and dissolution in the gastric environment. In this sense, a binder performs the opposite function of a disintegrant. The types of binders that can be used in drug formulations include gelatins of numerous grades, starches and starch derivatives (including corn starch, StaRx 1500, carboxymethylated starch), cellulose derivatives, polyvinylpyrollidones, Veegums, polyethylene glycols, sugars, e.g., sucrose and lactose, sodium alginate and waxes.

The fillers used to bulk up a drug tablet or other form also should not interfere with the tablet's dissolution. Numerous fillers include the starch derivatives, sugars (e.g., lactose and sucrose), sorbitol, mannitol, cellulose derivatives and their inorganic salts, corn starch, Starch 1500, calcium phosphate, and Avicel.

Likewise, lubricants aid in the machining of a drug tablet. Every tablet needs a lubricant so that it will be ejected from the machine die with minimum force. However, the lubricant also must not interfere with the dissolution of the tablet. Lubricants include waxes, fatty acids, sodium salts of fatty acids and stearates.

These objectives of this invention will be further understood with reference to the following nonlimiting examples.

### EXAMPLE 1

### Formation of Emulsion Preconcentrates of Perillyl Alcohol and Paclitaxel

Formulation A and B. 72.2 milligrams of paclitaxel were dissolved in 209 grams of perillyl alcohol by mixing at room temperature for 20 to 30 minutes. Separately, Cremophore and polyethylene glycol 300 were mixed for 15 minutes and added to the perillyl alcohol paclitaxel. 123 grams of d-alpha-tocopherol polyethylene glycol was added to form the final paclitaxel preconcentrate. The preconcentrate was assayed for stability over time by monitoring the content of paclitaxel and perillyl alcohol by HPLC. The stable preconcentrate was diluted in water (1:100) and particle size was monitored over time at 0° C., 4° C., and room temperature. In addition, presence or absence of paclitaxel crystals was measured microscopically.

Formulation B was made in a similar method as described in Table 1.

Formulation C. 68 milligrams of paclitaxel was dissolved in 193 milligrams of perillyl alcohol. 111 milligrams of Pluronic F 68 (BASF Corporation) were mixed with 404 milligrams of polyethylene glycol 300 and then added to the paclitaxel/perillyl alcohol solution. Finally, 223 milligrams of d-alpha-tocopherol polyethylene glycol were added to form the final preconcentrate. Stability was tested as for formulation A.

Formulation D was made in a similar method to b1 as shown in Table 1, except that cyclosporin A was added to the initial perillyl alcohol paclitaxel solution.

TABLE 1

| | Preconcentrate Formulation | | | |
|---|---|---|---|---|
| Component | A | B | C | D |
| Paclitaxel | 72 mgs | 10 mgs | 67 mgs | 9 mgs |
| Perillyl alcohol | 210 mgs | 125 mgs | 193 mgs | 114 mgs |
| Cremophore EL | 148 mgs | — | — | 319 mgs |
| Polyethylene glycol 300 | 452 mgs | 488 mgs | 404 mgs | 399 mgs |
| d-alpha-tocopherol | 123 mgs | 125 mgs | 222 mgs | 114 mgs |
| polyethylene glycol | | | | |
| Pluronic F 68 | — | 250 mgs | 111 mgs | — |
| Cyclosporin A | — | — | — | 45 mgs |

### EXAMPLE 2

### Bioavailability of Paclitaxel Following Intraduodenal Administration in Rats

Sprague-Dawley rats (approximately weighing 120 grams each) were catheterized surgically with jugular and duodenal catheters. Each group of rats, 3 animals per group, were given 9 micrograms/KG of paclitaxel either in formulation B or formulation D. Blood samples were collected at 0, 20, 40,60, 90, 120, and 240 minutes following administration of the formulations. The time 0 blood collection was obtained approximately 15 minutes before experimental application of formulations. Plasma samples were analyzed by a solid phase extraction of paclitaxel followed by HPLC. Pharmacokinetic parameters were calculated from the data using WinNonLin software (Pharsight). Approximately 100 ng paclitaxel per ml was observed in the plasma of each of the rats at 4 hours post administration. Absorption was equivalent in the rats given formulation a2 as with rats given formulation c, containing Cyclosporin A, an inhibitor of the P glycoprotein.

### EXAMPLE 3

### Effect of Emulsions on Human Breast Cancer Cell Lines

Human breast cancer cell lines are implanted subcutaneously into nude mice. Three human cell lines, MCF-7, BT-20, and MDA-MB-231 are used. Tumors are harvested and cells are grown in RPMI supplemented with fetal bovine serum (10%), ampicillin (100 micrograms per ml), streptomycin, (100 micrograms per ml), and glutamine (0.3%). The cells are grown to approximately 80% confluence and treated with paclitaxel in Cremophor (commercial formulations from Bristol Myers Squibb), Cremophor alone, dilution of perillyl alcohol preconcentrate formulations without paclitaxel, or paclitaxel in perillyl alcohol submicron formulation. Viable cells are determined at times after addition by enumerating proportion of living cells by dye exclusion technique using tetrazolium blue.

### EXAMPLE 4

### Tumor Regression by Oral Administration of Paclitaxel as a Preconcentrate

Athymic nude mice are injected subcutaneously with approximately 10⁷ MDA-MB-231 cells. Tumors develop at the injection site until they are approximately 100 mm³ in size. Mice are treated by intraperitoneal injection of cremophor paclitaxel or cremophor alone as controls. Subject mice are given doses of paclitaxel in emulsion preconcentrate ranging from 0 to 6 mgs/kg by oral gavage once a day. Tumor size is measured and proportion of mice with tumor regression is measured.

What is claimed is:

1. A pharmaceutical composition comprising:
(a) a monoterpene or a derivative thereof,
(b) one or more surfactants, and optionally
(c) one or more cosolvents.

2. The composition of claim 1, wherein said composition is self-emulsifying upon dilution with an aqueous solution or biological fluid.

3. The composition of claim 1, wherein said composition is an emulsion preconcentrate.

4. The composition of claim 1, wherein said composition is in the form of a microemulsion preconcentrate.

5. The composition of claim 1, wherein the monoterpene has antineoplastic activity.

6. The composition of claim 1, wherein the monoterpene is perillyl alcohol.

7. The composition of claim 1, wherein the monoterpene is perillic acid.

8. The composition of claim 1, wherein the monoterpene is perillaldehyde.

9. The composition of claim 1, further comprising one or more therapeutic agents.

10. The composition of claim 9, wherein the therapeutic agent is soluble in the monoterpene.

11. The composition of claim 9, wherein the therapeutic agent has aqueous solubility of less than 1 mg/ml, preferably less than 0.1 mg/ml.

12. The composition of claim 1, wherein the surfactant is an emulsifying agent selected from the group consisting of an alkyl glycerolphosphoryl choline, a polyoxyethylene polymer, a block copolymer of polyoxyethylene and polyoxypropylene, and an ethoxylated glycerol ester.

13. The composition of claim 6, wherein the perillyl alcohol is present at 1%-50% of the total weight of the composition.

14. The composition of claim 6, wherein the perillyl alcohol is present at 5%-40% of the total weight of the composition.

15. The composition of claim 6, wherein the perillyl alcohol is present at 5%-20% of the total weight of the composition.

16. The composition of claim 6, wherein the perillyl alcohol is present at 5%-10% of the total weight of the composition.

17. The composition of claims 1 or 12, wherein the surfactant is present at 1%-75% of the total weight of.the composition.

18. The composition of claims 1 or 12, wherein the surfactant is present at 10%-60% of the total weight of the composition.

19. The composition of claims 1 or 12, wherein the surfactant is present at 20%-50% of the total weight of the composition.

20. The composition of claim 9, wherein the therapeutic agent is an anti-cancer agent.

21. The composition of claim 20, wherein the therapeutic agent is a taxane.

22. The composition of claim 21, wherein the therapeutic agent is paclitaxel.

23. The composition of claim 22, wherein the paclitaxel comprises from about 1% to about 20% of the total weight of the composition.

24. The composition of claim 22, wherein the paclitaxel comprises from about 1% to 10% of the total weight of the composition.

25. The composition of claim 20, wherein the therapeutic agent is a topoisomerase inhibitor.

26. The composition of claim 25, wherein the topoisomerase inhibitor is selected from the group consisting of etoposide, camptothecin, topotecan, or a derivative thereof.

27. The composition according to any one of claim 1-16, 20-24, or 25, further comprising an inhibitor of P glycoprotein.

28. The composition of claim 17, further comprising an inhibitor of P glycoprotein.

29. The composition of claim 18, further comprising an inhibitor of P glycoprotein.

30. The composition of claim 19, further comprising an inhibitor of P glycoprotein.

31. The composition according to claim 27, 28 or 29 wherein the inhibitor of P glycoprotein is selected from the group consisting of cyclosporin A, ketoconazole, verapamil, or derivatives thereof.

32. The composition of claim 1, wherein the surfactant is an inhibitor of P-glycoprotein.

33. The composition of claim 32, wherein said inhibitor is selected from the group consisting of polyoxyethylene-polyoxypropylene block copolymer, a polysorbate, and α-tocopherol-polyethylene glycol-succinate.

34. The composition of claim 1, wherein the cosolvent is a polyhydric alcohol.

35. A composition of claim 34 wherein the polyhydric alcohol is selected from the group consisting of glycerol, sorbitol, mannitol, ethylene glycol, propylene glycol, polyethylene glycol and mixtures thereof.

36. The composition of claim 35, wherein the polyhydric alcohol is a polyethylene glycol.

37. A composition of claim 36, wherein the polythylene glycol has an average molecular weight in the range of about 100 to about 10,000 daltons

38. A composition of claim 36, wherein the polythylene glycol has an average molecular weight in the range of about 100 to about 1,000 daltons.

39. A composition of claim 36, wherein the polythylene glycol has an average molecular weight in the range of about 200 to about 600 daltons.

40. A method of treating a patient comprising oral administration of a composition according to any one of claims 1-16, 20-26, 28-30, 32-38, or 39.

41. A method of treating a patient comprising oral administration of a composition according to claim 17.

42. A method of treating a patient comprising oral administration of a composition according to claim 18.

43. A method of treating a patient comprising oral administration of a composition according to claim 19.

44. A method of treating a patient comprising oral administration of a composition according to claim 27.

45. A method of treating a patient comprising oral administration of a composition according to claim 31.

46. A method of treating a patient comprising oral administration of a composition according to any one of claims 1-16, 20-26, 28-30, 32-38, or 39, said composition further comprising paclitaxel, at a dose of paclitaxel from about 10 mg/m² to about 1000 mg/m².

47. A method of treating a patient comprising oral administration of a composition according to claim 17, said composition further comprising paclitaxel, at a dose of paclitaxel from about 10 mg/m² to about 1000 mg/m².

48. A method of treating a patient comprising oral administration of a composition according to claim 18, said composition further comprising paclitaxel, at a dose of paclitaxel from about 10 mg/m² to about 1000 mg/m².

49. A method of treating a patient comprising oral administration of a composition according to claim 19, said composition further comprising paclitaxel, at a dose of paclitaxel from about 10 mg/m² to about 1000 mg/m².

50. A method of treating a patient comprising oral administration of a composition according to claim 27, said composition further comprising paclitaxel, at a dose of paclitaxel from about 10 mg/m² to about 1000 mg/m².

51. A method of treating a patient comprising oral administration of a composition according to claim 31, said composition further comprising paclitaxel, at a dose of paclitaxel from about 10 mg/m² to about 1000 mg/m².

52. A method of treating a patient comprising oral administration of a composition according to any one of claims 1-16, 20-26, 28-30, 32-38, or 39, wherein said composition is contained in a form selected from the group consisting of contained in a soft gelatin capsule, a hard gelatin capsule, an enteric coated capsule, with flavoring agents and taste masking agents, or a tablet.

53. A method of treating a patient comprising oral administration of a composition according to claim 17, wherein said composition is contained in a form selected from the group consisting of contained in a soft gelatin capsule, a hard gelatin capsule, an enteric coated capsule, with flavoring agents and taste masking agents, or a tablet.

54. A method of treating a patient comprising oral administration of a composition according to claim 18, wherein said composition is contained in a form selected from the group consisting of contained in a soft gelatin capsule, a hard gelatin capsule, an enteric coated capsule, with flavoring agents and taste masking agents, or a tablet.

55. A method of treating a patient comprising oral administration of a composition according to claim 19, wherein said composition is contained in a form selected from the group consisting of contained in a soft gelatin capsule, a hard gelatin capsule, an enteric coated capsule, with flavoring agents and taste masking agents, or a tablet.

56. A method of treating a patient comprising oral administration of a composition according to claim 27, wherein said composition is contained in a form selected from the group consisting of contained in a soft gelatin capsule, a hard gelatin capsule, an enteric coated capsule, with flavoring agents and taste masking agents, or a tablet.

57. A method of treating a patient comprising oral administration of a composition according to claim 31, wherein said composition is contained in a form selected from the group consisting of contained in a soft gelatin capsule, a hard gelatin capsule, an enteric coated capsule, with flavoring agents and taste masking agents, or a tablet.

58. A method of forming a fine emulsion comprising mixing the composition according to any one of claims 1-16, 20-26, 28-30, 32-38, or 39 with a hydrophilic phase.

59. A method of forming a fine emulsion comprising mixing the composition according to claim 17 with a hydrophilic phase.

60. A method of forming a fine emulsion comprising mixing the composition according to claim 18 with a hydrophilic phase.

61. A method of forming a fine emulsion comprising mixing the composition according to claim 19 with a hydrophilic phase.

62. A method of forming a fine emulsion comprising mixing the composition according to claim 27 with a hydrophilic phase.

63. A method of forming a fine emulsion comprising mixing the composition according to claim 31 with a hydrophilic phase.

64. The method according to claim 53, wherein the fine emulsion has a mean droplet diameter of about 10 nm to 5000 nm.

65. The method according to claim 53, wherein the fine emulsion has a mean droplet diameter of about 10 nm to 1000 nm.

66. The method according to any one of claims 53 or 59, wherein the hydrophilic phase is an aqueous solution

67. A method of forming a fine emulsion in vivo comprising administering a composition according to any one of claims 1-16, 20-26, 28-30, 32-38, or 39, said composition mixing with biological fluids.

68. A method of forming a fine emulsion in vivo comprising administering a composition according to claim 17, said composition mixing with biological fluids.

69. A method of forming a fine emulsion in vivo comprising administering a composition according to claim 18, said composition mixing with biological fluids.

70. A method of forming a fine emulsion in vivo comprising administering a composition according to claim 19, said composition mixing with biological fluids.

71. A method of forming a fine emulsion in vivo comprising administering a composition according to claim 27, said composition mixing with biological fluids.

72. A method of forming a fine emulsion in vivo comprising administering a composition according to claim 31, said composition mixing with biological fluids.

73. The method according to claim 67, wherein the biological fluids are gastrointestinal fluids.

74. A method for increasing the absorption of a lipophilic therapeutic agent comprising administering a composition according to any one of claims 1-16, 20-26, 28-30, 32-38, or 39 to the mucosa.

75. A method for increasing the absorption of a lipophilic therapeutic agent comprising administering a composition according to claim 17 to the mucosa.

76. A method for increasing the absorption of a lipophilic therapeutic agent comprising administering a composition according to claim 18 to the mucosa.

77. A method for increasing the absorption of a lipophilic therapeutic agent comprising administering a composition according to claim 19 to the mucosa.

78. A method for increasing the absorption of a lipophilic therapeutic agent comprising administering a composition according to claim 27 to the mucosa.

79. A method for increasing the absorption of a lipophilic therapeutic agent comprising administering a composition according to claim 31 to the mucosa.

80. A method of increasing the absorption of a lipophilic therapeutic agent by inhibiting the action of P glycoprotein comprising administering a composition according to any one of claims 1-16, 20-26, 28-30, 32-38, or 39 to the mucosa.

81. A method of increasing the absorption of a lipophilic therapeutic agent by inhibiting the action of P glycoprotein comprising administering a composition according to claim 17 to the mucosa.

82. A method of increasing the absorption of a lipophilic therapeutic agent by inhibiting the action of P glycoprotein comprising administering a composition according to claim 18 to the mucosa.

83. A method of increasing the absorption of a lipophilic therapeutic agent by inhibiting the action of P glycoprotein comprising administering a composition according to claim 19 to the mucosa.

84. A method of increasing the absorption of a lipophilic therapeutic agent by inhibiting the action of P glycoprotein comprising administering a composition according to claim 27 to the mucosa.

85. A method of increasing the absorption of a lipophilic therapeutic agent by inhibiting the action of P glycoprotein comprising administering a composition according to claim 31 to the mucosa.
